(19) United States
(12) Patent Application Publication
(10) Pub. No.: US 2024/0131103 A1
(43) Pub. Date: Apr. 25, 2024

(54) NOVEL ARTHRITIS EMULGEL COMPOSITION AND ITS PREPARATION PROCESS

(71) Applicants: LYRUS LIFE SCIENCES PVT LTD, Bangalore (IN); NOKHA TRADING LLP, Bangalore (IN)

(72) Inventors: Chandanmal Pukhraj Bothra, Bangalore (IN); Hemanth Kumar Bothra, Bangalore (IN); Elayaraja Natarajan, Bangalore (IN); Jyolsna Agnes Jose, Bangalore (IN); Shailendra Thakur, Bangalore (IN)

(21) Appl. No.: 18/277,006

(22) PCT Filed: Feb. 17, 2022
(86) PCT No.: PCT/IB2022/051406

§ 371 Date: Aug. 11, 2023

(30) Foreign Application Priority Data
Feb. 18, 2021 (IN) 202141006775

(51) Int. Cl. A61K 36/324; A61K 9/06; A61K 9/107; A61K 31/045; A61K 31/12; A61K 31/618; A61K 31/7008; A61K 31/737; A61K 36/9068; A61K 47/10; A61K 47/18; A61K 47/24; A61K 47/32

(52) U.S. Cl. CPC A61K 36/324; A61K 9/06; A61K 9/107; A61K 31/045; A61K 31/12; A61K 31/618; A61K 31/7008; A61K 31/737; A61K 36/9068; A61K 47/10; A61K 47/183; A61K 47/24; A61K 47/32

(57) ABSTRACT

The present invention relates to the novel topical composition of arthritis emulgel. The present invention specifically relates to novel topical composition of arthritis emulgel comprising combination of active ingredients and pharmaceutically acceptable excipients. The present invention more specifically relates to novel composition of arthritis emulgel comprising combination of Curcumin (using Force C3™ augmented absorption technology), Boswellia serrata extract, Methyl salicylate, Menthol as active ingredients and pharmaceutically acceptable excipients. The present invention more specifically relates to novel composition, wherein composition optionally contains one or more of Ginger extract, Glucosamine sulphate and Chondroitin sulphate. The present invention also relates to process for the preparation of novel topical composition of arthritis emulgel comprising the steps of dissolving, mixing, adding, sonicating, adjusting the pH and stirring.

### FIELD OF INVENTION

The present invention relates to the novel topical composition of arthritis emulgel.

The present invention specifically relates to novel topical composition of arthritis emulgel comprising combination of active ingredients and pharmaceutically acceptable excipients.

The present invention more specifically relates to novel composition of arthritis emulgel comprising combination of Curcumin, Boswellia serrata, Methyl salicylate, Menthol as active ingredients and pharmaceutically acceptable excipients.

The present invention more specifically relates to novel composition, wherein composition optionally contains one or more of Ginger, Glucosamine sulphate and Chondroitin sulphate.

The present invention also relates to process for the preparation of novel topical composition of arthritis emulgel comprising the steps of dissolving, mixing, adding, sonicating, adjusting the pH and stirring.

### BACKGROUND OF INVENTION

Arthritis is a common condition that causes pain and inflammation (swelling) of the joints and bones. The main symptoms of arthritis include pain, stiffness, restricted movements of the joints, inflammation and swelling, warmth and redness of the skin over the joint. The most prevalent forms being osteoarthritis, rheumatoid arthritis, ankylosing spondylitis in adults and juvenile chronic arthritis (JCA) in children. Other forms of arthritis (e.g., systemic lupus erythematosus, scleroderma, Sjogren's syndrome, psoriatic arthritis, gout) are less common.

Arthritis is caused by continuous inflammation, which is a result from a complex series of actions and/or reactions triggered by the body's immunological response to tissue damage. Moderate inflammation is necessary for the healing process; however, continuous inflammation may lead to chronic conditions like arthritis and its associated pain.

### Curcumin

Curcumin is a bright yellow chemical produced by Curcuma longa plants. It is the principal curcuminoid of turmeric (Curcuma longa), a member of the ginger family, Zingiberaceae. Curcumin helps to reduce inflammation. Several studies suggest that it might ease symptoms of osteoarthritis and rheumatoid arthritis, like pain and inflammation. Curcumin, chemically described as (1E, 6E)-1,7-bis(4-hydroxy-3-methoxyphenyl)-1,6-heptadien-3,5-dione. It is a yellowish crystalline, odorless powder, poorly soluble in water, petroleum ether, and benzene, soluble in ethyl alcohols, glacial acetic acid and in propylene glycol, very soluble in acetone and ethyl ether.

Curcuminoids are phenolic compounds extracted from the dried rhizomes of Curcuma longa (Turmeric) having potent anti-inflammatory and anti-oxidant properties. Curcumin drops having 95% Curcuminoids (Curcumin, Demethoxycurcumin & Bisdemethoxycurcumin) & non-curcuminoids prepared using patented Force C³™ augmented absorption technology. It is soluble and bioavailable form providing potent anti-inflammatory and pain relieving effects.

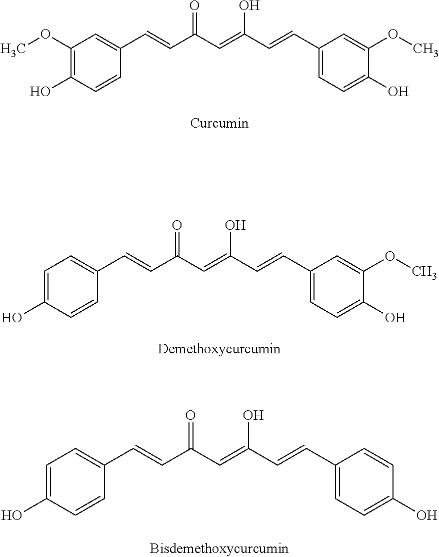

Curcumin inhibits NF-kB signaling pathways a potential therapeutic target in osteoarthritis and rheumatoid arthritis. Curcumin also inhibits COX-2, 5-LOX and TNF-alpha providing potent anti-inflammatory effects in Arthritis. Curcumin acts as an anti-inflammatory by inhibiting cyclooxygenase 2 (COX-2), inducible nitric oxide synthase (ins) and lipoxygenase (COX). INOs, LOX, and COX are key enzymes that mediate inflammatory processes.

Boswellia serrata Extract

Boswellia serrata is a medium to large branching tree, generally found in dry hilly areas of India, North Africa and the Middle East, belongs to the family Burseraceae. Boswellia serrata is called Indian olibanum, Indian frankincense or “Dhup”, it is also known as Salai guggul, and Sallaki in Sanskrit.

Gum-resin extracts of Boswellia serrata have been traditionally used in folk medicine for centuries to treat various chronic inflammatory diseases. The resinous part of Boswellia serrata possesses monoterpenes, diterpenes, triterpenes, tetracyclic triterpenic acids and four major pentacyclic triterpenic acids i.e. β-boswellic acid, acetyl-β-boswellic acid. 11-keto-β-boswellic acid and acetyl-11-keto-β-boswellic acid, responsible for inhibition of pro-inflammatory enzymes. Out of these four boswellic acids, acetyl-11-keto-β-boswellic acid is the most potent inhibitor of 5-lipoxygenase, an enzyme responsible for inflammation.

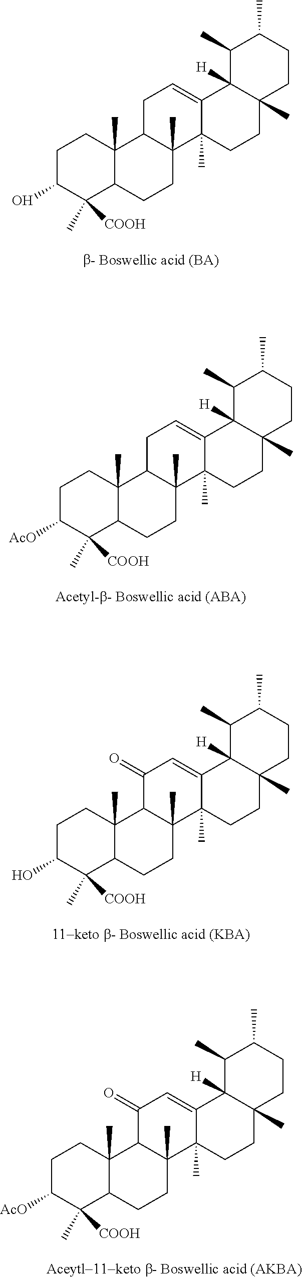

Boswellia serrata plant contains boswellic acid as its major active constituent which is present as α-boswellic acid; β-boswellic acid; 3-acetyl-11-keto β-boswellic acid (AKBA), responsible for anti-arthritic activity. From ancient times Boswellia serrata is being used in the management of rheumatoid arthritis, osteoarthritis solely because of these potent active constituents. Boswellic acid shows its activity by inhibiting the synthesis of pro-inflammatory cytokines and 5-lipoxygenase activity.

Ginger (Zingiber officinale Rosc.)

Non-steroidal anti-inflammatory drugs (NSAIDs) are generally considered to be the most effective treatment for inflammation and pain. However, the adverse effects associated with their use may outweigh the benefits for many patients, especially those suffering from long-term chronic conditions such as osteoarthritis. Considering the potentially significant gastrointestinal and cardiovascular risks of NSAID use, increasing numbers of patients are searching for alternative forms of pain management, which meet their needs in terms of pain amelioration and minimal adverse effects. Zingiber officinale, commonly known as ginger, has a long tradition of medicinal use as an anti-inflammatory agent for musculoskeletal diseases in Ayurvedic and Chinese medicine. Z. officinale is a member of the Zingiberaceae plant family, native to southern Asia, consisting of 49 genera and 1,300 species, 80-90 of which are Zingiber. It is a complex mixture of pharmacological compounds containing several hundred known constituents, including gingerols, beta-carotene, capsaicin, caffeic acid, curcumin, and salicylate Z. officinale possesses anti-emetic, positive inotropic, and carminative properties to promote secretion of saliva and gastric juices and to inhibit platelet aggregation. Several of its chemical constituents, including gingerols, shogaols, paradols, and zingerone, have demonstrated anti-inflammatory actions in vitro, inhibiting leukotriene synthesis, the activity of cyclooxygenase enzymes (COX-11 and COX-2), production of interleukins (Il-1 and Il-12), and tumor necrosis factor alpha in activated macrophages. In addition, it has been suggested that Z. officinale and its constituents—particularly shogaols—have agonize vallinoid (capsaicin) receptors TRPV1, which are involved in the central and peripheral processing of noxious stimuli.

There is a growing literature that has focused on assessing the value of the analgesic and anti-inflammatory properties of Z. officinale in human participants, including a recent review that evaluated the effectiveness of Z. officinale in the management of osteoarthritis.

Methyl Salicylate

Methyl salicylate or wintergreen oil or oil of wintergreen can be described as a natural ester produced by different plant species, specifically wintergreens. The plant species Gaultheria procumbens was first used in 1843 for extracting and isolating this compound. Methyl salicylate, this sweet-smelling member of the aspirin family is one of the most widely used counterirritants. Methyl salicylate has anti-inflammatory properties and is still used, incorporated into liniments and ointments for joint and muscle pains and for rheumatic conditions.

The chemical name for Methyl salicylate is 2-Hydroxybenzoic acid methyl ester. Methyl salicylate has a chemical formula of C₈H₈O₃ and a molecular mass of 152.15 g/mol. It has a structural formula of:

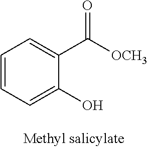 [SMILES: COC(=O)c1ccccc1O]

Menthol

Menthol is an organic compound made synthetically or obtained from the oils of corn mint, peppermint, or other mints. Preferably, Menthol is synthesized from peppermint oil according to the present invention. It is a waxy, crystalline substance, clear or white in color, which is solid at room temperature and melts slightly above. The main form of Menthol occurring in nature is (—)-Menthol, which is assigned the (1R,2S,5R) configuration. Menthol has local anesthetic and counter irritant qualities. Menthol is used for the treatment, control, prevention of pain in shoulder joint, pain in arthritis, pain in muscle strains or sprains, arthritic pain, pain in tendons, back pain, bruising, cramping etc.

The chemical name of Menthol is (1a,2b,5a)-5-Methyl-2-(1-methylethyl)cyclohexanol. Menthol has a chemical formula of C₁₀H₂₀O and a molecular mass of 156.27 g/mol. It has a structural formula of:

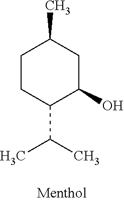

Glucosamine Sulphate

Glucosamine is an amino sugar and a prominent precursor in the biochemical synthesis of glycosylated proteins and lipids. Glucosamine is part of the structure of two polysaccharides, chitosan and chitin. Glucosamine is one of the most abundant monosaccharides. Glucosamine plays an important role in preserving joint health. Glucosamine seems to be able to help reduce the inflammation and pain associated with arthritis in your joints, possibly by helping to rebuild damaged cartilage and slow the progressive loss of joint space that occurs in the disease.

Glucosamine is an amino hexose sugar. The chemical name for Glucosamine is 2-Amino-2-deoxy-D-glucose. Glucosamine has a chemical formula of C₆H₁₃NO₅ and a molecular mass of 179.17 g/mol. It has a structural formula of:

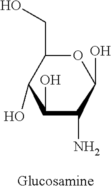

Glucosamine plays a vital role in building and repairing cartilage.

Glucosamine sulfate is a naturally occurring chemical found in the human body. It is in the fluid around joints. Glucosamine also exists in other places in nature. For example, Glucosamine sulfate used in dietary supplements is often Obtained from the shells of shellfish. Glucosamine sulfate used in dietary supplements does not always come from natural sources. It can also be made in a laboratory.

Chondroitin Sulphate

Chondroitin sulfate is a sulfated glycosaminoglycan (GAG) composed of a chain of alternating sugars (N-acetylgalactosamine and glucuronic acid). It is usually found attached to proteins as part of a proteoglycan. A chondroitin chain can have over 100 individual sugars, each of which can be sulfated in variable positions and quantities. Chondroitin sulfate is an important structural component of cartilage and provides much of its resistance to compression.

Chondroitin sulfate (CS) being a natural glycosaminoglycan is found in the cartilage and extracellular matrix. It shows clinical benefits in symptomatic osteoarthritis (OA) of the finger, knee, hip joints, low back, facial joints and other diseases due to its anti-inflammatory activity. Chondroitin sulfate is also called as Chondroitin sulfuric acid. It has a structural formula of:

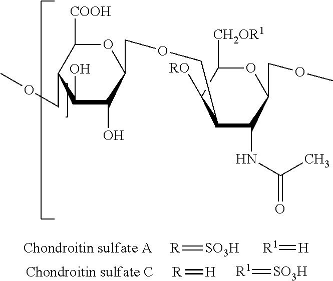

U.S. Pat. No. 5,853,753 A discloses liposomal compositions for the treatment of pain, composition comprising, a phospholipid, lecithin and surfactant, sodium chlorite without any pharmaceutical agents for the application via either dermally or subcutaneously.

U.S. Pat. No. 6,165,500 A discloses the method of transporting medical agents through the skin comprising the steps which include preparing transfersomes by containing lipid and surfactant with the specific weight ratio upon applying suitable amount of transfersomes in medium onto the skin.

U.S. Pat. No. 6,579,543 B1 discloses composition for topical application to an human skin for relief from a variety of symptoms caused by medical conditions or physical injuries. This patent discloses use of Curcumin, Boswellia, Glucosamine, Chondroitin sulfate and Menthol as alternate active ingredients. However, the combination use of all the active ingredients is not disclosed.

US Publication No. 2012/0220669 A1 discloses method for treating pain or inflammation or osteoarthritis comprising administering to a subject a formulation comprising one or more phospholipids and one or more surfactants, wherein the formulation does not comprise a non-lipid non-surfactant pharmaceutically active agent that has been approved for the treatment of pain, inflammation, or osteoarthritis.

US Publication No. 2015/0065461 A1 discloses method of treatment for pain, reduced mobility, associated with or more of a loss of lubrication, a loss of structural integrity, or swelling of a collagen structure or both comprising administering a vesicular formulation comprising a phospholipid and a surfactant by topically applied to skin surrounding the collagen structure.

US Publication No. 2015/0125407 A1 discloses method of administering biologically active agent by topical application of the biologically active agent and topically applying vesicular formulation after applying the biologically active agent wherein vesicular formulation comprising phospholipid and surfactant. This application does not disclose combination of Curcumin, Boswellia Serrata, Glucosamine, Chondroitin sulphate, Methyl salicylate and Menthol.

US Publication No. 2020/0061002 A1 discloses topical composition comprising one or more topical active ingredients, one or more nonionic surfactants and one or more viscosity enhancers. This application does not disclose combination of Curcumin, Boswellia Serrata, Glucosamine and Chondroitin sulphate.

All the prior art references relates to the use of Curcumin, Boswellia Serrata, Ginger extract, Glucosamine, Chondroitin sulphate, Methyl salicylate and Menthol for treating arthritis or liposomal compositions for the treatment of pain without any pharmaceutical agents or different topical compositions. However, none of the above prior-art document discloses synergistic combination of the present invention.

The inventors of the present invention provide novel composition of arthritis emulgel comprising the combination of Curcumin, Boswellia serrata extract, Methyl Salicylate and Menthol, as active ingredients and pharmaceutically acceptable excipients. The inventors of present invention also provide process for the preparation of novel topical composition of arthritis emulgel comprising the steps of dissolving, mixing, adding, sonicating, homogenizing, adjusting the pH and stirring.

### OBJECTIVE OF INVENTION

The main objective of the present invention is to provide novel composition of arthritis emulgel.

Another objective of the present invention is to provide novel composition of arthritis emulgel comprising the combination of active ingredients and pharmaceutically acceptable excipients.

Another objective of the present invention is to provide novel composition of arthritis emulgel comprising combination of Curcumin, Boswellia Serrata, Methyl Salicylate, Menthol as active ingredients and pharmaceutically acceptable excipients.

Yet another objective of the present invention is to provide novel composition wherein composition optionally contains one or more of Ginger, Glucosamine sulphate, Chondroitin sulphate.

Still another objective of the present invention is to provide process for the preparation of novel topical composition of arthritis emulgel comprising the steps of dissolving, mixing, adding, sonicating, homogenizing, adjusting the pH and stirring.

Another objective of the present invention is to provide novel composition of arthritis emulgel comprising combination of Curcumin, Boswellia Serrata, Methyl Salicylate, Menthol as active ingredients and pharmaceutically acceptable excipients, which is stable, provides better efficacy for a patient suffering arthritis.

Yet another objective of the present invention is to provide relief from moderate pain, symptoms of arthritis by the administration of topical administration of arthritis emulgel composition of the present invention.

### SUMMARY OF INVENTION

Accordingly, the present invention provides a novel composition of arthritis emulgel useful in relieving moderate pain and symptoms of arthritis.

One embodiment of the present invention provides a novel composition of arthritis emulgel comprising the combination of active ingredients and pharmaceutically acceptable excipients.

Another embodiment of the present invention provides a novel composition of arthritis emulgel comprising combination of Curcumin (using Force C3™ augmented absorption technology), Boswellia Serrata extract, Methyl salicylate and Menthol as active ingredients and pharmaceutically acceptable excipients.

Another embodiment of the present invention provides a novel composition of arthritis emulgel wherein the composition optionally contains one or more of Ginger, Glucosamine sulphate, Chondroitin Sulphate.

Another embodiment of the present invention provides a novel composition of arthritis emulgel comprising combination of Curcumin drops, Boswellia serrata extract, Methyl salicylate, Menthol as active ingredients; skin permeation enhancer, antimicrobial preservative, gelling agent, chelating agent, wetting agent, antioxidant, emulsifier, stabilizer, solvent and optionally emollient as pharmaceutically acceptable excipients.

Another embodiment of the present invention involves use of Curcumin drops produced using Force C3™ augmented absorption technology.

Another embodiment of the present invention provides a process for the preparation of novel topical composition of arthritis emulgel comprising the steps of dissolving, mixing, adding, sonicating, homogenizing, adjusting the pH and stirring.

Yet another embodiment of the present invention provides a novel composition of arthritis emulgel comprising:
(a) 3% to 10% (w/w) of active ingredients,
(b) 2% to 8% (w/w) of skin permeation enhancer,
(c) 0.5% to 3% (w/w) of preservative,
(d) 0.5% to 5% (w/w) of gelling agent,
(e) 0.01% to 1% (w/w) of chelating agent,
(f) 1% to 5% (w/w) of wetting agent,
(g) 0.01% to 1% (w/w) of antioxidants,
(h) 0.5% to 5% (w/w) of emulsifier,
(i) 1% to 5% (w/w) of stabilizer, and optionally
(j) 0.5% to 3% (w/w) of emollient.

Another embodiment of the present invention provides a novel composition of arthritis emulgel comprising combination of Curcumin drops, Boswellia serrata extract, Methyl salicylate, Menthol as active ingredients, phosphatidyl choline as skin permeation enhancer, benzyl alcohol as antimicrobial preservative, carboxypolymethylene, sodium CMC, hydroxypropyl methylcellulose, hydroxyethyl cellulose as gelling agent, EDTA sodium as chelating agent, polyoxyl castor oil as wetting agent, butylated hydroxy anisole and butylated hydroxytoluene as antioxidants, cetostearyl alcohol, sodium methyl cocoyl taurate and sodium lauroyl sarcosinate as emulsifier, triethanolamine as stabilizer, ethanol as solvent and propylene glycol as emollient.

Another embodiment of the present invention provides a novel composition of arthritis emulgel comprising combination of Curcumin drops, Boswellia serrata extract, Methyl salicylate, Menthol, Glucosamine sulphate and Chondroitin sulphate as active ingredients, phosphatidyl choline as skin permeation enhancer, benzyl alcohol as antimicrobial preservative, carboxypolymethylene, sodium CMC, hydroxypropyl methylcellulose, hydroxyethyl cellulose as gelling agent, EDTA sodium as chelating agent, polyoxyl castor oil as wetting agent, butylated hydroxy anisole and butylated hydroxytoluene as antioxidants, cetostearyl alcohol, sodium methyl cocoyl taurate and sodium lauroyl sarcosinate as emulsifier, triethanolamine as stabilizer, ethanol as solvent and propylene glycol as emollient.

Another embodiment of the present invention provides a novel composition of arthritis emulgel comprising combination of Curcumin drops, Boswellia serrata extract, Ginger extract, Methyl salicylate, Menthol, Glucosamine sulphate and Chondroitin sulphate as active ingredients, phosphatidyl choline as skin permeation enhancer, benzyl alcohol as antimicrobial preservative, carboxypolymethylene, sodium CMC, hydroxypropyl methylcellulose, hydroxyethyl cellulose as gelling agent, EDTA sodium as chelating agent, polyoxyl castor oil as wetting agent, butylated hydroxy anisole and butylated hydroxytoluene as antioxidants, cetostearyl alcohol, sodium methyl cocoyl taurate and sodium lauroyl sarcosinate as emulsifier, triethanolamine as stabilizer, ethanol as solvent and propylene glycol as emollient.

Yet another embodiment of the present invention provides a novel composition of arthritis emulgel comprising:
(a) 3% to 10% (w/w) of combination of Curcumin drops, Boswellia serrata extract, Methyl salicylate and Menthol,
(b) 2% to 8% (w/w) of phosphatidyl choline,
(c) 0.5% to 3% (w/w) of benzyl alcohol,
(d) 0.5% to 5% (w/w) of gelling agent,
(e) 0.01% to 1% (w/w) of EDTA sodium,
(f) 1% to 5% (w/w) of polyoxyl castor oil,
(g) 0.01% to 1% (w/w) of butylated hydroxy anisole and butylated hydroxytoluene,
(h) 0.5% to 5% (w/w) of emulsifier,
(i) 1% to 5% (w/w) of triethanolamine,
(j) 0.5% to 3% (w/w) of ethanol, and optionally
(k) 0.5% to 3% (w/w) of propylene glycol.

Yet another embodiment of the present invention provides a novel composition of arthritis emulgel comprising:
(a) 3% to 10% (w/w) of combination of Curcumin drops, Boswellia serrata extract, Ginger extract, Methyl salicylate and Menthol,
(b) 2% to 8% (w/w) of phosphatidyl choline,
(c) 0.5% to 3% (w/w) of benzyl alcohol,
(d) 0.5% to 5% (w/w) of gelling agent,
(e) 0.01% to 1% (w/w) of EDTA sodium,
(f) 1% to 5% (w/w) of polyoxyl castor oil,
(g) 0.01% to 1% (w/w) of butylated hydroxy anisole and butylated hydroxytoluene,
(h) 0.5% to 3% (w/w) of emulsifier,
(i) 1% to 5% (w/w) of triethanolamine, and
(j) 0.5% to 3% (w/w) of ethanol,

Yet another embodiment of the present invention provides a novel composition of arthritis emulgel comprising:
(a) 3% to 10% (w/w) of combination of Curcumin drops, Boswellia serrata extract, Methyl salicylate, Menthol and Glucosamine sulphate,
(b) 2% to 8% (w/w) of phosphatidyl choline,
(c) 0.5% to 3% (w/w) of benzyl alcohol,
(d) 0.5% to 5% (w/w) of gelling agent,
(e) 0.01% to 1% (w/w) of EDTA sodium,
(f) 1% to 5% (w/w) of polyoxyl castor oil,
(g) 0.01% to 1% (w/w) of butylated hydroxy anisole and butylated hydroxytoluene,
(h) 0.5% to 3% (w/w) of emulsifier,
(i) 1% to 5% (w/w) of triethanolamine,
(j) 0.5% to 3% (w/w) of ethanol, and optionally
(k) 0.5% to 3% (w/w) of propylene glycol.

Yet another embodiment of the present invention provides a novel composition of arthritis emulgel comprising:
(a) 3% to 10% (w/w) of combination of Curcumin drops, Boswellia serrata extract, Methyl salicylate, Menthol and Chondroitin sulphate,
(b) 2% to 8% (w/w) of phosphatidyl choline,
(c) 0.5% to 3% (w/w) of benzyl alcohol,
(d) 0.5% to 5% (w/w) of gelling agent,
(e) 0.01% to 1% (w/w) of EDTA sodium,
(f) 1% to 5% (w/w) of polyoxyl castor oil,
(g) 0.01% to 1% (w/w) of butylated hydroxy anisole and butylated hydroxytoluene,
(h) 0.5% to 3% (w/w) of emulsifier,
(i) 1% to 5% (w/w) of triethanolamine,
(j) 0.5% to 3% (w/w) of ethanol, and optionally
(k) 0.5% to 3% (w/w) of propylene glycol.

Yet another embodiment of the present invention provides a novel composition of arthritis emulgel comprising:
(a) 3% to 10% (w/w) of combination of Curcumin drops, Boswellia serrata extract, Methyl salicylate, Menthol, Glucosamine sulphate, Chondroitin sulphate,
(b) 2% to 8% (w/w) of phosphatidyl choline,
(c) 0.5% to 3% (w/w) of benzyl alcohol,
(d) 0.5% to 5% (w/w) of gelling agent,
(e) 0.01% to 1% (w/w) of EDTA sodium,
(f) 1% to 5% (w/w) of polyoxyl castor oil,
(g) 0.01% to 1% (w/w) of butylated hydroxy anisole and butylated hydroxytoluene,
(h) 0.5% to 3% (w/w) of emulsifier,
(i) 1% to 5% (w/w) of triethanolamine,
(j) 0.5% to 3% (w/w) of ethanol, and optionally
(k) 0.5% to 3% (w/w) of propylene glycol.

Yet another embodiment of the present invention provides a novel composition of arthritis emulgel comprising:
(a) 3% to 10% (w/w) of combination of Curcumin drops, Boswellia serrata extract, Ginger extract, Methyl salicylate, Menthol, Glucosamine sulphate, Chondroitin sulphate,
(b) 2% to 8% (w/w) of phosphatidyl choline,
(c) 0.5% to 3% (w/w) of benzyl alcohol,
(d) 0.5% to 5% (w/w) of gelling agent,
(e) 0.01% to 1% (w/w) of EDTA sodium,
(f) 1% to 5% (w/w) of polyoxyl castor oil,
(g) 0.01% to 1% (w/w) of butylated hydroxy anisole and butylated hydroxytoluene,
(h) 0.5% to 3% (w/w) of emulsifier,
(i) 1% to 5% (w/w) of triethanolamine,
(j) 0.5% to 3% (w/w) of ethanol, and optionally
(k) 0.5% to 3% (w/w) of propylene glycol.

In yet another embodiment, the present invention provides a novel process for the preparation of composition of arthritis emulgel, wherein the process comprising steps of:
(a) dissolving phosphatidyl choline, Boswellia serrata in ethanol under stirring and checking the clarity of the solution after each addition,
(b) mixing emulsifier with butylated hydroxytoluene (BHT) and butylated hydroxy anisole (BHA) and heating the mixture to about 45-50° C. in water bath,
(c) adding contents of step (b) to step (a) under stirring,
(d) adding polyoxyl castor oil, disodium EDTA in water and warming slightly to dissolve under continuous stirring,
(e) adding contents of step (d) to step (c) under sonication and continue the stirring until forms uniform white emulsion,
(f) dispersing gelling agent into water under stirring,
(g) adding contents of step (e) to step (f) under stirring to get uniform white liquid emulsion,
(h) dissolving benzyl alcohol and Menthol crystals in Methyl salicylate,
(i) adding contents of step (h) to step (g) under homogenization to get uniform white liquid emulsion and checking pH before neutralization,
(j) adding triethanolamine in a very thin stream to step (i) under stirring till pH reaches to about 6.80 to 7.00, continuing to mix for at least 15 minutes after triethanolamine addition for obtaining a smooth white gel with uniform pH and viscosity build up, and
(k) adding Curcumin drops prepared using Force C³™ augmented absorption technology under stirring for obtaining a smooth yellowish emulgel.

In yet another embodiment, the present invention provides a novel process for the preparation of composition of arthritis emulgel, wherein the process comprising steps of:
(a) dissolving phosphatidyl choline, Boswellia serrata in ethanol under stirring,
(b) dissolving Ginger extract in small quantity of ethanol and adding to the above solution,
(c) mixing emulsifier with butylated hydroxytoluene (BHT) and butylated hydroxy anisole (BHA) and heating the mixture to about 45-50° C. in water bath,
(d) adding contents of step (c) to step (b) under stirring,
(e) adding polyoxyl castor oil, disodium EDTA in water and warming slightly to dissolve under continuous stirring,
(f) adding contents of step (e) to step (d) under sonication and continue the stirring until forms uniform white emulsion,
(g) dispersing gelling agent into water under stirring,
(h) adding contents of step (f) to step (g) under stirring to get uniform white liquid emulsion,
(i) dissolving benzyl alcohol and Menthol crystals in Methyl salicylate,
(j) adding contents of step (i) to step (h) under homogenization to get uniform white liquid emulsion and checking pH before neutralization,
(k) adding triethanolamine in a very thin stream to step (j) under stirring till pH reaches to about 6.80 to 7.00, continuing to mix for at least 15 minutes after triethanolamine addition for obtaining a smooth white gel with uniform pH and viscosity build up, and
(l) adding Curcumin drops prepared using Force C₃™ augmented absorption technology under stirring for obtaining a smooth yellowish emulgel.

In yet another embodiment, the present invention provides a novel process for the preparation of composition of arthritis emulgel, wherein the process comprising steps of:
(a) dissolving phosphatidyl choline, Boswellia serrata in ethanol under stirring and checking the clarity of the solution after each addition,
(b) mixing emulsifier with butylated hydroxytoluene (BHT) and butylated hydroxy anisole (BHA) and heating the mixture to about 45-50° C. in water bath,
(c) adding contents of step (b) to step (a) under stirring,
(d) adding polyoxyl castor oil, disodium EDTA in water and warming slightly to dissolve under continuous stirring,
(e) adding contents of step (d) to step (c) under stirring,
(f) dissolving Chondroitin sulphate and Glucosamine in water one after another,
(g) adding contents of step (f) to step (e) under stirring and sonicating to get uniform white liquid emulsion,
(h) dispersing gelling agent into water under stirring,
(i) adding contents of step (g) to step (h) under stirring to get uniform white liquid emulsion,
(j) dissolving benzyl alcohol and Menthol crystals in Methyl salicylate,
(k) adding contents of step (j) to step (i) under homogenization to get uniform white liquid emulsion and checking pH before neutralization,
(l) adding propylene glycol under homogenization,
(m) adding triethanolamine in a very thin stream to step (l) under stirring till pH reaches to about 6.80 to 7.00, continuing to mix for at least 15 minutes after triethanolamine addition for obtaining a smooth white gel with uniform pH and viscosity build up, and
(n) adding Curcumin drops prepared using Force C³™ augmented absorption technology under stirring for obtaining a smooth yellowish emulgel.

In yet another embodiment, the present invention provides a novel process for the preparation of composition of arthritis emulgel, wherein the process comprising steps of:
(a) dissolving phosphatidyl choline, Boswellia serrata in ethanol under stirring and checking the clarity of the solution after each addition,
(b) dissolving Ginger extract in small quantity of ethanol and adding to the above solution,
(c) mixing emulsifier with butylated hydroxytoluene (BHT) and butylated hydroxy anisole (BHA) and heating the mixture to about 45-50° C. in water bath,
(d) adding contents of step (c) to step (b) under stirring,
(e) adding polyoxyl castor oil, disodium EDTA in water and warming slightly to dissolve under continuous stirring,
(f) adding contents of step (e) to step (d) under stirring,
(g) dissolving Chondroitin sulphate and Glucosamine in water one after another,
(h) adding contents of step (g) to step (f) under stirring and sonicating to get uniform white liquid emulsion,
(i) dispersing gelling agent into water under stirring,
(j) adding contents of step (h) to step (i) under stirring to get uniform white liquid emulsion,
(k) dissolving benzyl alcohol and Menthol crystals in Methyl salicylate,
(l) adding contents of step (k) to step (j) under homogenization to get uniform white liquid emulsion and checking pH before neutralization,
(m) adding triethanolamine in a very thin stream to step (l) under stirring till pH reaches to about 6.80 to 7.00, continuing to mix for at least 15 minutes after triethanolamine addition for obtaining a smooth white gel with uniform pH and viscosity build up, and
(n) adding Curcumin drops prepared using Force C³™ augmented absorption technology under stirring for obtaining a smooth yellowish emulgel.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

The present invention provides novel arthritis emulgel composition comprising combinations of active ingredients and pharmaceutically acceptable excipients.

The present invention provides novel arthritis emulgel composition comprising combination of Curcumin, Boswellia serrata, Methyl salicylate, Menthol as active ingredients, skin permeation enhancer, antimicrobial preservative, gelling agent, chelating agent, wetting agent, antioxidant, emulsifier, stabilizer, solvent and optionally emollient as pharmaceutically acceptable excipients.

Another embodiment of the present invention provides novel composition of arthritis emulgel wherein composition optionally contains one or more of Ginger, Glucosamine sulphate, Chondroitin sulphate.

The term “active ingredients” of the present invention is used to relieve from moderate pain, symptoms of arthritis by the topical administration of arthritis emulgel. Preferably used active ingredients are Curcumin drops, Boswellia serrata, Ginger, Methyl salicylate, Menthol, Glucosamine sulphate and Chondroitin sulphate.

Curcumin, as used herein can be in the form of powder, drops, extract, preferably as drops.

Curcumin drops are prepared by using Force C³™ augmented absorption technology. The concentration of Curcumin drops used in the arthritis emulgel is from 0.00001% to 0.003% (w/w) and most preferably used concentration of Curcumin drops is 0.00225% (w/w) of the total composition.

Boswellia serrata, as used herein can be in the form of powder, drops, extract, preferably as extract.

The concentration of Boswellia serrata extract used in the arthritis emulgel is from 0.01% to 0.3% (w/w) and most preferably used concentration of Boswellia serrata extract is 0.1% (w/w) of the total composition.

Ginger, as used herein can be in the form of powder, drops, extract, preferably as extract.

The concentration of Ginger (Zingiber officinale) extract used in the arthritis emulgel is from 0.01% to 0.3% (w/w) and most preferably used concentration of Ginger (Zingiber officinale) extract is 0.01% (w/w) of the total composition.

The concentration of Methyl salicylate used in the arthritis emulgel is from 2% to 8% (w/w) and most preferably used concentration of Methyl salicylate is 5% (w/w) of the total composition.

The concentration of Menthol used in the arthritis emulgel is from 0.5% to 3% (w/w) and most preferably used concentration of Menthol is 1% (w/w) of the total composition.

The concentration of Glucosamine sulphate used in the arthritis emulgel is from 0.004% to 0.006% (w/w) and most preferably used concentration of Glucosamine is from % to 0.005% (w/w) of the total composition.

The concentration of Chondroitin sulfate used in the arthritis emulgel is from 0.1% to 0.5% (w/w) and most preferably used concentration of Chondroitin sulfate is 0.2% (w/w) of the total composition.

Skin permeation enhancer of the present invention includes but not limited to lecithin, lysolecithin, hydroxylated lecithin, lysophosphatidylcholine, is phosphatidylcholine, phosphatidylethanolamine, phosphatidylserine, didecanoyl-L-α-phosphatidylcholine, laurolylcarnitine, acylcarnitine, palmitoyl-D,L-carnitine. Preferably used skin permeation enhancer is phosphatidylcholine obtained from soy.

The concentration of skin permeation enhancer e used in the arthritis emulgel is from 2% to 8% (w/w) and most preferably used concentration of phosphatidylcholine is 5% (w/w) of the total composition.

Antimicrobial preservative used in the composition of the present invention includes but not limited to methylparaben, butylparaben, propylparaben, benzyl alcohol, sorbic acid, imidurea, thimerisal, propyl gallate, citric acid, disodium edetate, and the like. Preferably used antimicrobial preservative is benzyl alcohol.

The concentration of preservative used in the arthritis emulgel is from 0.5% to 3% (w/w) and most preferably used concentration of preservative is 1% (w/w) of the total composition.

Gelling agent used in the composition of the present invention includes but not limited to sodium CMC, carbomer like poly(acrylic acid (carbomer), cellulose derivatives, especially cellulose ethers, such as alkyl- and hydroxyalkyl cellulose, for instance ethylcellulose, methylcellulose, hydroxyethyl cellulose, hydroxypropyl methylcellulose, hydroxypropyl cellulose, especially ethyl(hydroxyethyl) cellulose (EHEC), carboxy methyl cellulose, other polysaccharides and derivatives, such as naturally derived polysaccharides and derivatives thereof, including modified Carrageenan and synthetic polymers such as polyethylene glycols, polyethylene oxides, polyvinyl pyrrolidones and poly acrylic acid. Preferably used gelling agents are poly(acrylic acid (carbomer), sodium CMC, hydroxypropyl methylcellulose and hydroxyethyl cellulose.

The concentration of gelling agent used in the arthritis emulgel is from 0.5% to 5% (w/w) and most preferably used concentration of gelling agent is from 1% to 3% (w/w) of the total composition.

Chelating agent used in the composition of the present invention includes but not limited to ethylenediamine tetraacetic acid (EDTA), EDTA sodium, diammonium EDTA, dipotassium EDTA, calcium disodium EDTA, HEDTA, TEA-EDTA, is tetrasodium EDTA, tripotassium EDTA, trisodium phosphate, diammonium citrate, galactaric acid, galacturonic acid, gluconic acid, glucuronic acid, humic acid, cyclodextrin, potassium citrate, potassium EDTMP, sodium citrate, sodium EDTMP, and mixtures thereof. Preferably used chelating agent is EDTA sodium.

The concentration of chelating agent used in the arthritis emulgel is from 0.01% to 1% (w/w) and most preferably used concentration of chelating agent is from 0.1% (w/w) of the total composition.

Wetting agent used in the composition of the present invention includes but not limited to castor oil derivatives like Kolliphor EL, polyoxyl castor oil, cholesterol, polyglycolyzed glycerides, acetylated monoglycerides, sorbitan fatty acid esters, poloxamers, polysorbates, polyoxyethylene sorbitan fatty acid esters, polyoxyethylene compounds, monoglycerides or ethoxylated derivatives thereof, diglycerides or polyoxyethylene derivatives thereof, sodium docusate, sodium laurylsulfate, cholic acid or derivatives thereof, ethoxylated alcohols, ethoxylated esters, ethoxylated amides, polyoxypropylene compounds, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, SPAN's (e.g., sorbitan esters), TWEEN's (i.e., sucrose esters), glucose (dextrose) esters, alkali metal sulfates, quaternary ammonium compounds, amidoamines, and aminimides, simethicone, lecithins, alcohols, phospholipids, and mixtures. Preferably used wetting agent is polyoxyl castor oil.

The concentration of wetting agent used in the arthritis emulgel is from 1% to 5% (w/w) and most preferably used concentration of wetting agent is from 3.5% (w/w) of the total composition.

Antioxidant used in the composition of the present invention includes but not limited to butylated hydroxytoluene (BHT), butylated hydroxyanisole (BHA), ascorbyl linoleate, ascorbyl dipalmitate, ascorbyl tocopherol maleate, calcium ascorbate, carotenoids, kojic acid, thioglycolic acid, tocopherol, tocopherol acetate, tocophereth-5, tocophereth-12, tocophereth-18, tocophereth-80, and mixtures thereof. Preferably used antioxidants are butylated hydroxy anisole and butylated hydroxytoluene.

The concentration of antioxidant used in the arthritis emulgel is from 0.01% to 1% (w/w) and most preferably used concentration of antioxidant individually is from 0.02% to 0.2% (w/w) and concentration of antioxidant in combination is 0.22% (w/w).

Emulsifier of the present invention includes but not limited to cetostearyl alcohol, sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium methyl cocoyl taurate, sodium lauroyl glutamate, sodium myristoyl glutamate, and sodium cocoyl glutamate and mixtures thereof. Preferably used emulsifiers are cetostearyl alcohol, sodium methyl cocoyl taurate, sodium lauroyl sarcosinate.

The concentration of emulsifier used in the arthritis emulgel is from 0.5% to 5% (w/w) and most preferably used concentration of emulsifier is from 1.25% (w/w) of the total composition.

Stabilizer used in the composition of the present invention includes but not limited to sodium hydroxide, potassium hydroxide, ammonium hydroxide, monoethanolamine, diethanolamine, triethanolamine, diisopropanolamine, aminomethyl propanol, trimethamine, tetrahydroxypropyl ethylenediamine, citric acid. Preferably used stabilizer is triethanolamine.

The concentration of stabilizer used in the arthritis emulgel is from 1% to 5% (w/w) and most preferably used concentration of stabilizer is 2.4% (w/w) of the total composition.

Solvent used in the composition of the present invention includes but not limited to ethanol, isopropyl alcohol, glycofurol, polyethylene glycol (PEG 200, 400), glycerol, polypropylene glycol, propylene glycol, N-methyl-2-pyrolidone and ethyl alcohol or mixture thereof. Preferably used solvent is ethanol.

The concentration of solvent used in the arthritis emulgel is from 0.5% to 3% (w/w) and most preferably used concentration of solvent is 1.5% (w/w) of the total composition.

Emollient used in the composition of the present invention includes but not limited to polyhydric alcohols such as propylene glycol, butylene glycol, polyethylene glycol (e.g., polyethylene glycol 400), glycerol, and sorbitol; fatty acid triglycerides such as a mixture of caprylic and capric triglycerides (e.g., Crodamol™ GTCC-LQ, Captex®, Labrafac™ Lipophile WL), palmitic triglyceride, oleic triglyceride, caprylic triglyceride, capric triglyceride, and linoleic triglyceride; fatty acid esters such as isopropyl myristate, isopropyl palmitate, dibutyl adipate, and dibutyl phthalate; fatty acids such as oleic acid and stearic acid; oils such as mineral oil, lanolin oil, coconut oil, cocoa butter, olive oil, jojoba oil, and castor oil; cyclomethicone; hydrogenated lanolin; waxes; lecithin; and mixtures thereof. Preferably used emollient is propylene glycol.

The concentration of emollient used in the arthritis emulgel is from 0.5% to 3% (w/w) and most preferably used concentration of emollient is 1.25% (w/w) of the total composition.

Vehicle used in the composition of the present invention is purified water. The concentration of vehicle used in the arthritis emulgel is from 70% to 80% (w/w) and most preferably used concentration of solvent is 76.475% (w/w) of the total composition.

The present invention provides process for the preparation of novel topical composition of arthritis emulgel comprising the steps of dissolving, mixing, adding, sonicating, adjusting the pH and stirring.

The following examples describes the nature of the invention and are given only for the purpose of illustrating the present invention in more detail and are not limitative and relate to solutions, which have been particularly effective on bench scale.

### Example 1

| S. | | Concentration |
|---|---|---|
| No | Ingredients | (% w/w) |
| 1. | Curcumin drops* | 0.00225 |
| 2. | Boswellia serrata extract | 0.100 |
| 3. | Glucosamine sulphate | 0.005 |
| 4. | Chondroitin sulphate | 0.200 |
| 5. | Methyl salicylate | 5.000 |
| 6. | Menthol | 1.000 |
| 7. | Phosphatidylcholine | 5.000 |
| 8. | Benzyl alcohol | 1.000 |
| 9. | Ethanol | 1.500 |
| 10. | Carbomer | 1.000 |
| 11. | EDTA sodium | 0.100 |
| 12. | Polyoxyl castor oil | 3.500 |
| 13. | Butylated hydroxyanisole (BHA) | 0.020 |
| 14. | Butylated hydroxytoluene (BHT) | 0.200 |
| 15. | Cetostearyl alcohol | 1.250 |
| 16. | Triethanolamine | 2.400 |
| 17. | Purified water | 76.475 |
| | Total | 98.75 |

*Prepared using Force C^{3TM} augmented absorption technology.

### Manufacturing Process:

Phosphatidyl choline and Boswellia serrata extract were dissolved in Ethanol by placing in clean and dry manufacturing container and stirred. Clarity of the solution was checked after each addition. Cetostearyl alcohol, Butylated hydroxytoluene (BHT) and Butylated hydroxy anisole (BHA) were mixed and heated to about 45-50° C. in water bath to dissolve. Both the contents were added together and stirred. Polyoxyl castor oil and disodium EDTA were added to water, warmed slightly to dissolve and stirred. Contents were mixed with obtained mixture and stirred. Chondroitin sulphate and Glucosamine were dissolved in water one after another and stirred. Contents were added to the obtained mixture and sonicated to get uniform white liquid emulsion. Carbomer polymer was dispersed into water and stirred. Contents were added to the obtained mixture and stirred to get uniform white liquid emulsion. Benzyl alcohol and Menthol crystals were dissolved in Methyl salicylate and stirred. Contents were added to the obtained mixture and homogenized to get uniform white liquid emulsion and pH was checked. Triethanolamine was added in a very thin stream and stirred till pH reaches to about 6.80 to 7.00. Mixed continuously for at least 15 minutes for uniform pH, viscosity build up and smooth white gel was obtained. Curcumin drops was prepared using Force C³™ augmented absorption technology was added, stirred and smooth yellowish emulgel of combination of Curcumin, Boswellia serrata extract, Glucosamine sulphate, Chondroitin sulphate, Methyl salicylate and Menthol was obtained.

### Example 2

| S. | | Concentration |
|---|---|---|
| No | Ingredients | (% w/w) |
| 1. | Curcumin drops* | 0.00225 |
| 2. | Boswellia serrata Extract | 0.100 |
| 3. | Methyl salicylate | 5.000 |
| 4. | Menthol | 1.000 |
| 5. | Phosphatidylcholine | 5.000 |
| 6. | Benzyl alcohol | 1.000 |
| 7. | Ethanol | 1.500 |
| 8. | Carbomer | 1.000 |
| 9. | EDTA sodium | 0.100 |
| 10. | Polyoxyl castor oil | 3.500 |
| 11. | Butylated hydroxyanisole (BHA) | 0.020 |
| 12. | Butylated hydroxytoluene (BHT) | 0.200 |
| 13. | Cetostearyl alcohol | 1.250 |
| 14. | Triethanolamine | 2.400 |
| 15. | Purified water | 76.475 |
| | Total | 98.54 |

*Prepared using Force C^{3TM} augmented absorption technology.

### Manufacturing Process:

Phosphatidyl choline and Boswellia serrata extract were dissolved in Ethanol by placing in clean and dry manufacturing container and stirred. Clarity of the solution was checked after each addition. Cetostearyl alcohol, Butylated hydroxytoluene (BHT) and Butylated hydroxy anisole (BHA) were mixed and heated to about 45-50° C. in water bath to dissolve. Both the contents were added together and stirred. Polyoxyl castor oil and disodium EDTA were added to water, warmed slightly to dissolve and stirred. Contents were mixed with obtained mixture and stirred. Contents were added to the obtained mixture and sonicated to get uniform white liquid emulsion. Carbomer polymer was dispersed into water and stirred. Contents were added to the obtained mixture and stirred to get uniform white liquid emulsion. Benzyl alcohol and Menthol crystals were dissolved in Methyl salicylate and stirred. Contents were added to the obtained mixture and homogenized to get uniform white liquid emulsion and pH was checked. Triethanolamine was added in a very thin stream and stirred till pH reaches to about 6.80 to 7.00. Mixed continuously for at least 15 minutes for uniform pH, viscosity build up and smooth white gel was obtained. Curcumin drops was prepared using Force C³™ augmented absorption technology was added, stirred and smooth yellowish emulgel of combination of Curcumin, Boswellia serrata extract, Methyl salicylate and Menthol was obtained.

### Example 3

| S. | | Concentration |
|---|---|---|
| No | Ingredients | (% w/w) |
| 1. | Curcumin drops* | 0.00225 |
| 2. | Boswellia serrata extract | 0.100 |
| 3. | Glucosamine sulphate | 0.005 |
| 4. | Chondroitin sulphate | 0.200 |
| 5. | Methyl salicylate | 5.000 |
| 6. | Menthol | 1.000 |
| 7. | Phosphatidylcholine | 5.000 |
| 8. | Benzyl slcohol | 1.000 |
| 9. | Ethanol | 1.500 |
| 10. | Sodium CMC | 3.000 |
| 11. | EDTA sodium | 0.100 |
| 12. | Polyoxyl castor oil | 3.500 |
| 13. | Butylated hydroxyanisole (BHA) | 0.020 |
| 14. | Butylated hydroxytoluene (BHT) | 0.200 |
| 15. | Cetostearyl alcohol | 1.250 |
| 16. | Triethanolamine | 2.400 |
| 17. | Purified Water | 76.475 |
| | Total | 100.75 |

*Prepared using Force C^{3TM} augmented absorption technology.

### Manufacturing Process:

Phosphatidyl choline and Boswellia serrata extract were dissolved in Ethanol by placing in clean and dry manufacturing container and stirred. Clarity of the solution was checked after each addition. Cetostearyl alcohol, Butylated hydroxytoluene (BHT) and Butylated hydroxy anisole (BHA) were mixed and heated to about 45-50° C. in water bath to dissolve. Both the contents were added together and stirred. Polyoxyl castor oil and disodium EDTA were added to water, warmed slightly to dissolve and stirred. Contents were mixed with obtained mixture and stirred. Chondroitin sulphate and Glucosamine were dissolved in water one after another and stirred. Contents were added to the obtained mixture and sonicated to get uniform white liquid emulsion. Sodium CMC polymer was dispersed into water and stirred. Contents were added to the obtained mixture and stirred to get uniform white liquid emulsion. Benzyl alcohol and Menthol crystals were dissolved in Methyl salicylate and stirred. Contents were added to the obtained mixture and homogenized to get uniform white liquid emulsion and pH was checked. Triethanolamine was added in a very thin stream and stirred till pH reaches to about 6.80 to 7.00. Mixed continuously for at least 15 minutes for uniform pH, viscosity build up and smooth white gel was obtained. Curcumin drops was prepared using Force C³™ augmented absorption technology was added, stirred and smooth yellowish emulgel of combination of Curcumin, Boswellia serrata extract, Glucosamine sulphate, Chondroitin sulphate, Methyl salicylate and Menthol was obtained.

### Example 4

| S. | | Concentration |
|---|---|---|
| No | Ingredients | (% w/w) |
| 1. | Curcumin drops* | 0.00225 |
| 2. | Boswellia serrata extract | 0.100 |
| 3. | Glucosamine sulphate | 0.005 |
| 4. | Chondroitin sulphate | 0.200 |
| 5. | Methyl salicylate | 5.000 |
| 6. | Menthol | 1.000 |
| 7. | Phosphatidylcholine | 5.000 |
| 8. | Benzyl alcohol | 1.000 |
| 9. | Ethanol | 1.500 |
| 10. | Hydroxypropyl methylcellulose | 2.500 |
| 11. | EDTA sodium | 0.100 |
| 12. | Polyoxyl castor oil | 3.500 |
| 13. | Butylated hydroxyanisole (BHA) | 0.020 |
| 14. | Butylated hydroxytoluene (BHT) | 0.200 |
| 15. | Cetostearyl alcohol | 1.250 |
| 16. | Triethanolamine | 2.400 |
| 17. | Purified water | 76.475 |
| | Total | 100.25 |

*Prepared using Force C^{3TM} augmented absorption technology.

### Manufacturing Process:

Phosphatidyl choline and Boswellia serrata extract were dissolved in Ethanol by placing in clean and dry manufacturing container and stirred. Clarity of the solution was checked after each addition. Cetostearyl alcohol, Butylated hydroxytoluene (BHT) and Butylated hydroxy anisole (BHA) were mixed and heated to about 45-50° C. in water bath to dissolve. Both the contents were added together and stirred. Polyoxyl castor oil and disodium EDTA were added to water, warmed slightly to dissolve and stirred. Contents were mixed with obtained mixture and stirred. Chondroitin sulphate and Glucosamine were dissolved in water one after another and stirred. Contents were added to the obtained mixture and sonicated to get uniform white liquid emulsion. Hydroxypropyl methylcellulose was dispersed into water and stirred. Contents were added to the obtained mixture and stirred to get uniform white liquid emulsion. Benzyl alcohol and Menthol crystals were dissolved in Methyl salicylate and stirred. Contents were added to the obtained mixture and homogenized to get uniform white liquid emulsion and pH was checked. Triethanolamine was added in a very thin stream and stirred till pH reaches to about 6.80 to 7.00. Mixed continuously for at least 15 minutes for uniform pH, viscosity build up and smooth white gel was obtained. Curcumin drops was prepared using Force C³™ augmented absorption technology was added, stirred and smooth yellowish emulgel of combination of Curcumin, Boswellia serrata extract, Glucosamine sulphate, Chondroitin sulphate, Methyl salicylate and Menthol was obtained.

### Example 5

| S. | | Concentration |
|---|---|---|
| No | Ingredients | (% w/w) |
| 1. | Curcumin drops* | 0.00225 |
| 2. | Boswellia serrata extract | 0.100 |
| 3. | Glucosamine sulphate | 0.005 |
| 4. | Chondroitin sulphate | 0.200 |
| 5. | Methyl salicylate | 5.000 |
| 6. | Menthol | 1.000 |
| 7. | Phosphatidylcholine | 5.000 |
| 8. | Benzyl alcohol | 1.000 |
| 9. | Ethanol | 1.500 |
| 10. | Hydroxyethyl cellulose | 1.000 |
| 11. | EDTA sodium | 0.100 |
| 12. | Polyoxyl castor oil | 3.500 |
| 13. | Butylated hydroxyanisole (BHA) | 0.020 |
| 14. | Butylated hydroxytoluene (BHT) | 0.200 |
| 15. | Cetostearyl alcohol | 1.250 |
| 16. | Triethanolamine | 2.400 |
| 17. | Purified water | 76.475 |
| | Total | 98.75 |

*Prepared using Force C^{3TM} augmented absorption technology.

### Manufacturing Process:

Phosphatidyl choline and Boswellia serrata extract were dissolved in Ethanol by placing in clean and dry manufacturing container and stirred. Clarity of the solution was checked after each addition. Cetostearyl alcohol, Butylated hydroxytoluene (BHT) and Butylated hydroxy anisole (BHA) were mixed and heated to about 45-50° C. in water bath to dissolve. Both the contents were added together and stirred. Polyoxyl castor oil and disodium EDTA were added to water, warmed slightly to dissolve and stirred. Contents were mixed with obtained mixture and stirred. Chondroitin sulphate and Glucosamine were dissolved in water one after another and stirred. Contents were added to the obtained mixture and sonicated to get uniform white liquid emulsion. Hydroxyethyl cellulose was dispersed into water and stirred. Contents were added to the obtained mixture and stirred to get uniform white liquid emulsion. Benzyl alcohol and Menthol crystals were dissolved in Methyl salicylate and stirred. Contents were added to the obtained mixture and homogenized to get uniform white liquid emulsion and pH was checked. Triethanolamine was added in a very thin stream and stirred till pH reaches to about 6.80 to 7.00. Mixed continuously for at least 15 minutes for uniform pH, viscosity build up and smooth white gel was obtained. Curcumin drops was prepared using Force C³™ augmented absorption technology was added, stirred and smooth yellowish emulgel of combination of Curcumin, Boswellia serrata extract, Glucosamine sulphate, Chondroitin sulphate, Methyl salicylate and Menthol was obtained.

### Example 6

| S. | | Concentration |
|---|---|---|
| No | Ingredients | (% w/w) |
| 1. | Curcumin drops* | 0.00225 |
| 2. | Boswellia serrata extract | 0.100 |
| 3. | Glucosamine sulphate | 0.005 |
| 4. | Chondroitin sulphate | 0.200 |
| 5. | Methyl salicylate | 5.000 |
| 6. | Menthol | 1.000 |
| 7. | Phosphatidylcholine | 5.000 |
| 8. | Benzyl alcohol | 1.000 |
| 9. | Ethanol | 1.500 |
| 10. | Carbomer | 1.000 |
| 11. | EDTA sodium | 0.100 |
| 12. | Polyoxyl castor oil | 3.500 |
| 13. | Butylated hydroxyanisole (BHA) | 0.020 |
| 14. | Butylated hydroxytoluene (BHT) | 0.200 |
| 15. | Cetostearyl alcohol | 1.250 |
| 16. | Triethanolamine | 2.400 |
| 17. | Propylene glycol | 1.25 |
| 18. | Purified water | 76.475 |
| | Total | 100.00 |

*Prepared using Force C^{3TM} augmented absorption technology.

### Manufacturing Process:

Phosphatidyl choline and Boswellia serrata extract were dissolved in Ethanol by placing in clean and dry manufacturing container and stirred. Clarity of the solution was checked after each addition. Cetostearyl alcohol, Butylated hydroxytoluene (BHT) and Butylated hydroxy anisole (BHA) were mixed and heated to about 45-50° C. in water bath to dissolve. Both the contents were added together and stirred. Polyoxyl castor oil and disodium EDTA were added to water, warmed slightly to dissolve and stirred. Contents were mixed with obtained mixture and stirred. Chondroitin sulphate and Glucosamine were dissolved in water one after another and stirred. Contents were added to the obtained mixture and sonicated to get uniform white liquid emulsion. Carbomer polymer was dispersed into water and stirred. Contents were added to the obtained mixture and stirred to get uniform white liquid emulsion. Benzyl alcohol and Menthol crystals were dissolved in Methyl salicylate and stirred. Contents were added to the obtained mixture and homogenized to get uniform white liquid emulsion. Propylene glycol was added under homogenization and pH was checked. Triethanolamine was added in a very thin stream and stirred till pH reaches to about 6.80 to 7.00. Mixed continuously for at least 15 minutes for uniform pH, viscosity build up and smooth white gel was obtained. Curcumin drops was prepared using Force C³™ augmented absorption technology was added, stirred and smooth yellowish emulgel of combination of Curcumin, Boswellia serrata extract, Glucosamine sulphate, Chondroitin sulphate, Methyl salicylate and Menthol was obtained.

### Example 7

| S. | | Concentration |
|---|---|---|
| No | Ingredients | (% w/w) |
| 1. | Curcumin drops* | 0.00225 |
| 2. | Boswellia serrata extract | 0.100 |
| 3. | Glucosamine sulphate | 0.005 |
| 4. | Chondroitin sulphate | 0.200 |
| 5. | Methyl salicylate | 5.000 |
| 6. | Menthol | 1.000 |
| 7. | Phosphatidylcholine | 5.000 |
| 8. | Benzyl alcohol | 1.000 |
| 9. | Ethanol | 1.500 |
| 10. | Carbomer | 1.000 |
| 11. | EDTA sodium | 0.100 |
| 12. | Polyoxyl castor oil | 3.500 |
| 13. | Butylated hydroxyanisole (BHA) | 0.020 |
| 14. | Butylated hydroxytoluene (BHT) | 0.200 |
| 15. | Sodium methyl cocoyl taurate | 1.250 |
| 16. | Triethanolamine | 2.400 |
| 17. | Purified water | 76.475 |
| | Total | 98.75 |

*Prepared using Force C^{3TM} augmented absorption technology.

### Manufacturing Process:

Phosphatidyl choline and Boswellia serrata extract were dissolved in Ethanol by placing in clean and dry manufacturing container and stirred. Clarity of the solution was checked after each addition. Sodium methyl cocoyl taurate, Butylated hydroxytoluene (BHT) and Butylated hydroxy anisole (BHA) were mixed and heated to about 45-50° C. in water bath to dissolve. Both the contents were added together and stirred. Polyoxyl castor oil and disodium EDTA were added to water, warmed slightly to dissolve and stirred. Contents were mixed with obtained mixture and stirred. Chondroitin sulphate and Glucosamine were dissolved in water one after another and stirred. Contents were added to the obtained mixture and sonicated to get uniform white liquid emulsion. Carbomer polymer was dispersed into water and stirred. Contents were added to the obtained mixture and stirred to get uniform white liquid emulsion. Benzyl alcohol and Menthol crystals were dissolved in Methyl salicylate and stirred. Contents were added to the obtained mixture and homogenized to get uniform white liquid emulsion. Triethanolamine was added in a very thin stream and stirred till pH reaches to about 6.80 to 7.00. Mixed continuously for at least 15 minutes for uniform pH, viscosity build up and smooth white gel was obtained. Curcumin drops was prepared using Force C³™ augmented absorption technology was added, stirred and smooth yellowish emulgel of combination of Curcumin, Boswellia serrata extract, Glucosamine sulphate, Chondroitin Sulphate, Methyl Salicylate and Menthol was obtained.

### Example 8

| S. | | Concentration |
|---|---|---|
| No | Ingredients | (% w/w) |
| 1. | Curcumin Drops* | 0.00225 |
| 2. | Boswellia Serrata Extract | 0.100 |
| 3. | Glucosamine Sulphate | 0.005 |
| 4. | Chondroitin Sulphate | 0.200 |
| 5. | Methyl Salicylate | 5.000 |
| 6. | Menthol | 1.000 |
| 7. | Phosphatidylcholine | 5.000 |
| 8. | Benzyl Alcohol | 1.000 |
| 9. | Ethanol | 1.500 |
| 10. | Carbomer | 1.000 |
| 11. | EDTA Sodium | 0.100 |
| 12. | Polyoxyl castor oil | 3.500 |
| 13. | Butylated hydroxyanisole (BHA) | 0.020 |
| 14. | Butylated hydroxytoluene (BHT) | 0.200 |
| 15. | Sodium lauroyl sarcosinate | 1.250 |
| 16. | Triethanolamine | 2.400 |
| 17. | Purified Water | 76.475 |
| | Total | 98.75 |

*Prepared using Force C^{3TM} augmented absorption technology.

### Manufacturing Process:

Phosphatidyl choline and Boswellia serrata extract were dissolved in Ethanol by placing in clean and dry manufacturing container and stirred. Clarity of the solution was checked after each addition. Sodium lauroyl sarcosinate, Butylated hydroxytoluene (BHT) and Butylated hydroxy anisole (BHA) were mixed and heated to about 45-50° C. in water bath to dissolve. Both the contents were added together and stirred. Polyoxyl castor oil and disodium EDTA were added to water, warmed slightly to dissolve and stirred. Contents were mixed with obtained mixture and stirred. Chondroitin sulphate and Glucosamine were dissolved in water one after another and stirred. Contents were added to the obtained mixture and sonicated to get uniform white liquid emulsion. Carbopol® 980 NF polymer was dispersed into water and stirred. Contents were added to the obtained mixture and stirred to get uniform white liquid emulsion. Benzyl alcohol and Menthol crystals were dissolved in Methyl salicylate and stirred. Contents were added to the obtained mixture and homogenized to get uniform white liquid emulsion. Triethanolamine was added in a very thin stream and stirred till pH reaches to about 6.80 to 7.00. Mixed continuously for at least 15 minutes for uniform pH, viscosity build up and smooth white gel was obtained. Curcumin drops was prepared using Force C³™ augmented absorption technology was added, stirred and smooth yellowish emulgel of combination of Curcumin, Boswellia serrata extract, Glucosamine sulphate, Chondroitin sulphate, Methyl salicylate and Menthol was obtained.

### Example 9

| S. | | Concentration |
|---|---|---|
| No | Ingredients | (% w/w) |
| 1. | Curcumin drops* | 0.002 |
| 2. | Boswellia serrata extract | 0.150 |
| 3. | Ginger (Zingiber officinale) extract | 0.01 |
| 4. | Glucosamine sulphate | 0.005 |
| 5. | Chondroitin sulphate | 0.200 |
| 6. | Methyl salicylate | 5.000 |
| 7. | Menthol | 1.000 |
| 8. | Phosphatidylcholine | 5.000 |
| 9. | Benzyl alcohol | 1.000 |
| 10. | Ethanol | 1.500 |
| 11. | Carbomer | 1.000 |
| 12. | EDTA sodium | 0.100 |
| 13. | Polyoxyl castor oil | 3.500 |
| 14. | Butylated hydroxyanisole (BHA) | 0.020 |
| 15. | Butylated hydroxytoluene (BHT) | 0.200 |
| 16. | Cetostearyl alcohol | 1.250 |
| 17. | Triethanolamine | 2.400 |
| 18. | Purified water | 76.475 |
| | Total | 98.75 |

*Prepared using Force C^{3TM} augmented absorption technology.

### Manufacturing Process:

Phosphatidyl choline and Boswellia serrata extract were dissolved in Ethanol by placing in clean and dry manufacturing container and stirred. Dissolve Ginger extract in small quantity of ethanol and add to the above solution. Clarity of the solution was checked after each addition. Cetostearyl alcohol, Butylated hydroxytoluene (BHT) and Butylated hydroxy anisole (BHA) were mixed and heated to about 45-50° C. in water bath to dissolve. Both the contents were added together and stirred. Polyoxyl castor oil and disodium EDTA were added to water, warmed slightly to dissolve and stirred. Contents were mixed with obtained mixture and stirred. Chondroitin sulphate and Glucosamine were dissolved in water one after another and stirred. Contents were added to the obtained mixture and sonicated to get uniform white liquid emulsion. Carbomer polymer was dispersed into water and stirred. Contents were added to the obtained mixture and stirred to get uniform white liquid emulsion. Benzyl alcohol and Menthol crystals were dissolved in Methyl salicylate and stirred. Contents were added to the obtained mixture and homogenized to get uniform white liquid emulsion and pH was checked. Triethanolamine was added in a very thin stream and stirred till pH reaches to about 6.80 to 7.00. Mixed continuously for at least 15 minutes for uniform pH, viscosity build up and smooth white gel was obtained. Curcumin drops was prepared using Force C³™ augmented absorption technology was added, stirred and smooth yellowish emulgel of combination of Curcumin, Boswellia serrata extract, Ginger (Zingiber officinale) extract, Glucosamine sulphate, Chondroitin sulphate, Methyl salicylate and Menthol was obtained.

### Example 10

| S. | | Concentration |
|---|---|---|
| No | Ingredients | (% w/w) |
| 1. | Curcumin drops* | 0.00225 |
| 2. | Boswellia serrata extract | 0.10 |
| 3. | Ginger (Zingiber officinale) extract | 0.01 |
| 4. | Menthol | 1.000 |
| 5. | Methyl salicylate | 5.000 |
| 6. | Phosphatidylcholine | 5.000 |
| 7. | Benzyl alcohol | 1.000 |
| 8. | Ethanol | 1.500 |
| 9. | Carbomer | 1.000 |
| 10. | EDTA sodium | 0.100 |
| 11. | Polyoxyl castor oil | 3.500 |
| 12. | Butylated hydroxyanisole (BHA) | 0.020 |
| 13. | Butylated hydroxytoluene (BHT) | 0.200 |
| 14. | Cetostearyl alcohol | 1.250 |
| 15. | Triethanolamine | 2.400 |
| 16. | Purified water | 76.475 |
| | Total | 98.75 |

*Prepared using Force C^{3TM} augmented absorption technology.

### Manufacturing Process:

Phosphatidyl choline and Boswellia serrata extract were dissolved in Ethanol by placing in clean and dry manufacturing container and stirred. Dissolve Ginger extract in small quantity of ethanol and add to the above solution. Clarity of the solution was checked after each addition. Cetostearyl alcohol, Butylated hydroxytoluene (BHT) and Butylated hydroxy anisole (BHA) were mixed and heated to about 45-50° C. in water bath to dissolve. Both the contents were added together and stirred. Polyoxyl castor oil and disodium EDTA were added to water, warmed slightly to dissolve and stirred. Contents were mixed with obtained mixture and stirred Carbomer polymer was dispersed into water and stirred. Contents were added to the obtained mixture and stirred to get uniform white liquid emulsion. Benzyl alcohol and Menthol crystals were dissolved in Methyl salicylate and stirred. Contents were added to the obtained mixture and homogenized to get uniform white liquid emulsion and pH was checked. Triethanolamine was added in a very thin stream and stirred till pH reaches to about 6.80 to 7.00. Mixed continuously for at least 15 minutes for uniform pH, viscosity build up and smooth white gel was obtained. Curcumin drops was prepared using Force C³™ augmented absorption technology was added, stirred and smooth yellowish emulgel of combination of Curcumin, Boswellia serrata extract, Ginger (Zingiber officinale) Extract, Methyl salicylate and Menthol was obtained.

1. A novel composition of arthritis emulgel comprising Curcumin, Boswellia serrata, Methyl salicylate and Menthol as active ingredients and pharmaceutically acceptable excipients.

2. The composition as claimed in claim 1, wherein said arthritis emulgel comprising:
(a) 3% to 10% (w/w) of Curcumin, Boswellia serrata, Methyl salicylate and Menthol as active ingredients,
(b) 2% to 8% (w/w) of skin permeation enhancer,
(c) 0.5% to 3% (w/w) of preservative,
(d) 0.5% to 5% (w/w) of gelling agent,
(e) 0.01% to 1% (w/w) of chelating agent,
(f) 1% to 5% (w/w) of wetting agent,
(g) 0.01% to 1% (w/w) of antioxidants,
(h) 0.5% to 5% (w/w) of emulsifier,
(i) 1% to 5% (w/w) of stabilizer, and optionally
(j) 0.5% to 3% (w/w) of emollient.

3. The composition as claimed in claim 1, wherein Curcumin is Curcumin drops produced using Force C3™ augmented absorption technology.

4. The composition as claimed in claim 1, wherein the composition optionally contains one or more of Ginger, Glucosamine sulphate, Chondroitin sulphate and combination thereof.

5. The composition as claimed in claim 2, wherein the skin permeation enhancer is lecithin, lysolecithin, hydroxylated lecithin, lysophosphatidylcholine, phosphatidylcholine, phosphatidylethanolamine, phosphatidylserine, didecanoyl-L-α-phosphatidylcholine, laurolylcarnitine, acylcarnitine or palmitoyl-D,L-carnitine.

6. The composition as claimed in claim 2, wherein the preservative is methylparaben, butylparaben, propylparaben, benzyl alcohol, sorbic acid, imidurea, thimerisal, propyl gallate, citric acid, disodium edetate and combinations thereof.

7. The composition as claimed in claim 2, wherein the gelling agent is sodium CMC, carbomer like poly(acrylic acid (Carbomer), hydroxyalkyl cellulose, ethylcellulose, methylcellulose, hydroxyethyl cellulose, hydroxypropyl methylcellulose, hydroxypropyl cellulose, ethyl(hydroxyethyl) cellulose (EHEC), carboxy methyl cellulose, naturally derived polysaccharides and derivatives thereof, modified carrageenan, polyethylene glycols, polyethylene oxides, polyvinyl pyrrolidones, poly acrylic acid and combinations thereof.

8. The composition as claimed in claim 2, wherein the chelating agent is ethylenediamine tetraacetic acid (EDTA), EDTA sodium, diammonium EDTA, dipotassium EDTA, calcium disodium EDTA, HEDTA, TEA-EDTA, tetrasodium EDTA, tripotassium EDTA, trisodium phosphate, diammonium citrate, galactaric acid, galacturonic acid, gluconic acid, glucuronic acid, humic acid, cyclodextrin, potassium citrate, potassium EDTMP, sodium citrate, sodium EDTMP and combinations thereof.

9. The composition as claimed in claim 2, wherein the wetting agent is castor oil derivatives, polyoxyl castor oil, cholesterol, polyglycolyzed glycerides, acetylated monoglycerides, sorbitan fatty acid esters, poloxamers, polysorbates, polyoxyethylene sorbitan fatty acid esters, polyoxyethylene compounds, monoglycerides or ethoxylated derivatives thereof, diglycerides or polyoxyethylene derivatives thereof, sodium docusate, sodium laurylsulfate, cholic acid or derivatives thereof, ethoxylated alcohols, ethoxylated esters, ethoxylated amides, polyoxypropylene compounds, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, SPAN's (e.g., sorbitan esters), TWEEN's (i.e., sucrose esters), glucose (dextrose) esters, alkali metal sulfates, quaternary ammonium compounds, amidoamines, and aminimides, simethicone, lecithins, alcohols, phospholipids and combinations thereof.

10. The composition as claimed in claim 2, wherein the antioxidant is butylated hydroxytoluene (BHT), butylated hydroxyanisole (BHA), ascorbyl linoleate, ascorbyl dipalmitate, ascorbyl tocopherol maleate, calcium ascorbate, carotenoids, kojic acid, thioglycolic acid, tocopherol, tocopherol acetate, tocophereth-5, tocophereth-12, tocophereth-18, tocophereth-80 and combinations thereof.

11. The composition as claimed in claim 2, wherein the emulsifier is cetostearyl alcohol, sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium methyl cocoyl taurate, sodium lauroyl glutamate, sodium myristoyl glutamate, and sodium cocoyl glutamate and combinations thereof.

12. The composition as claimed in claim 2, wherein the stabilizer is sodium hydroxide, potassium hydroxide, ammonium hydroxide, monoethanolamine, diethanolamine, triethanolamine, diisopropanolamine, aminomethyl propanol, trimethamine, tetrahydroxypropyl ethylenediamine, citric acid and combinations thereof.

13. The composition as claimed in claim 2, wherein the emollient is propylene glycol, butylene glycol, polyethylene glycol, polyethylene glycol 400, glycerol, sorbitol, mixture of caprylic and capric triglycerides, palmitic triglyceride, oleic triglyceride, caprylic triglyceride, capric triglyceride, linoleic triglyceride, isopropyl myristate, isopropyl palmitate, dibutyl adipate, dibutyl phthalate, oleic acid, stearic acid, mineral oil, lanolin oil, coconut oil, cocoa butter, olive oil, jojoba oil, castor oil, cyclomethicone, hydrogenated lanolin, waxes, lecithin and combinations thereof.

14. The composition as claimed in claim 1, wherein the composition of arthritis emulgel comprising:
(a) 3% to 10% (w/w) of combination of Curcumin drops, Boswellia serrata extract, Methyl salicylate and Menthol,
(b) 2% to 8% (w/w) of phosphatidyl choline,
(c) 0.5% to 3% (w/w) of benzyl alcohol,
(d) 0.5% to 5% (w/w) of gelling agent,
(e) 0.01% to 1% (w/w) of EDTA sodium,
(f) 1% to 5% (w/w) of polyoxyl castor oil,
(g) 0.01% to 1% (w/w) of butylated hydroxy anisole and butylated hydroxytoluene,
(h) 0.5% to 5% (w/w) of emulsifier,
(i) 1% to 5% (w/w) of triethanolamine,
(j) 0.5% to 3% (w/w) of ethanol, and optionally
(k) 0.5% to 3% (w/w) of propylene glycol.

15. The composition as claimed in claim 1, wherein the composition of arthritis emulgel comprising:
(a) 3% to 10% (w/w) of combination of Curcumin drops, Boswellia serrata extract, Ginger extract, Methyl salicylate and Menthol,
(b) 2% to 8% (w/w) of phosphatidyl choline,
(c) 0.5% to 3% (w/w) of benzyl alcohol,
(d) 0.5% to 5% (w/w) of gelling agent,
(e) 0.01% to 1% (w/w) of EDTA sodium,
(f) 1% to 5% (w/w) of polyoxyl castor oil,
(g) 0.01% to 1% (w/w) of butylated hydroxy anisole and butylated hydroxytoluene,
(h) 0.5% to 5% (w/w) of emulsifier,
(i) 1% to 5% (w/w) of triethanolamine, and
(j) 0.5% to 3% (w/w) of ethanol.

16. The composition as claimed in claim 1, wherein the composition of arthritis emulgel comprising:
(a) 3% to 10% (w/w) of combination of Curcumin drops, Boswellia serrata extract, Methyl salicylate, Menthol, Glucosamine sulphate, Chondroitin sulphate,
(b) 2% to 8% (w/w) of phosphatidyl choline,
(c) 0.5% to 3% (w/w) of benzyl alcohol,
(d) 0.5% to 5% (w/w) of gelling agent,
(e) 0.01% to 1% (w/w) of EDTA sodium,
(f) 1% to 5% (w/w) of polyoxyl castor oil,
(g) 0.01% to 1% (w/w) of butylated hydroxy anisole and butylated hydroxytoluene,
(h) 0.5% to 3% (w/w) of emulsifier,
(i) 1% to 5% (w/w) of triethanolamine,
(j) 0.5% to 3% (w/w) of ethanol, and optionally
(k) 0.5% to 3% (w/w) of propylene glycol.

17. The composition as claimed in claim 1, wherein the composition of arthritis emulgel comprising:
(a) 3% to 10% (w/w) of combination of Curcumin drops, Boswellia serrata extract, Methyl salicylate, Ginger extract, Menthol, Glucosamine sulphate, Chondroitin sulphate,
(b) 2% to 8% (w/w) of phosphatidyl choline,
(c) 0.5% to 3% (w/w) of benzyl alcohol,
(d) 0.5% to 5% (w/w) of gelling agent,
(e) 0.01% to 1% (w/w) of EDTA sodium,
(f) 1% to 5% (w/w) of polyoxyl castor oil,
(g) 0.01% to 1% (w/w) of butylated hydroxy anisole and butylated hydroxytoluene,
(h) 0.5% to 3% (w/w) of emulsifier,
(i) 1% to 5% (w/w) of triethanolamine, and
(j) 0.5% to 3% (w/w) of ethanol.

18. The composition as claimed in claim 2, wherein the gelling agent is carboxypolymethylene or sodium CMC or hydroxypropyl methylcellulose or hydroxyethyl cellulose

19. The composition as claimed in claim 2, wherein the emulsifier is cetostearyl alcohol or sodium methyl cocoyl taurate or sodium lauroyl sarcosinate.

20. (canceled)

21. (canceled)

22. (canceled)

23. (canceled)

24. The process for the preparation of composition as claimed in claim 2, wherein the process comprising steps of:
(a) dissolving skin permeation enhancer, Boswellia serrata in ethanol under stirring and checking the clarity of the solution after each addition,
(b) mixing emulsifier with antioxidant and heating the mixture to about 45-50° C. in water bath,
(c) adding contents of step (b) to step (a) under stirring,
(d) adding wetting agent, chelating agent in water and warming slightly to dissolve under continuous stirring,
(e) adding contents of step (d) to step (c) under sonication and continue the stirring until forms uniform white emulsion,
(f) dispersing gelling agent into water under stirring,
(g) adding contents of step (e) to step (f) under stirring to get uniform white liquid emulsion,
(h) dissolving preservative and Menthol crystals in Methyl salicylate,
(i) adding contents of step (h) to step (g) under homogenization to get uniform white liquid emulsion and checking pH before neutralization,
(j) adding stabilizer in a very thin stream to step (i) under stirring till pH reaches to about 6.80 to 7.00, continuing to mix for at least 15 minutes after stabilizer addition for obtaining a smooth white gel with uniform pH and viscosity build up, and
(k) adding Curcumin drops prepared using Force C³™ augmented absorption technology under stirring for obtaining a smooth yellowish emulgel.
